SEQUENCE LISTING (19 sequences)

SEQ ID NO: 1 - protein, 453 aa, synthetic construct
  REGION 1..453: note = Synthetic
EVQLVESGGGLVQPGRSLRLSCVASGFTFNDYAMHWVRQAPGKGLEWVSVISWNSDSIGYADSVKGRFTISRDNAKNSLYLQMHSLRAEDTALYYCAKDNHYGSGSYYYYQYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 449 aa, synthetic construct
  REGION 1..449: note = Synthetic
EVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVASASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 3 - protein, 214 aa, synthetic construct
  REGION 1..214: note = Synthetic
EIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 127 aa, synthetic construct
  REGION 1..127: note = Synthetic
EVQLVESGGGLVQPGRSLRLSCVASGFTFNDYAMHWVRQAPGKGLEWVSVISWNSDSIGYADSVKGRFTISRDNAKNSLYLQMHSLRAEDTALYYCAKDNHYGSGSYYYYQYGMDVWGQGTTVTVSS

SEQ ID NO: 5 - protein, 123 aa, synthetic construct
  REGION 1..123: note = Synthetic
EVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVAS

SEQ ID NO: 6 - protein, 108 aa, synthetic construct
  REGION 1..108: note = Synthetic
EIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIKR

SEQ ID NO: 7 - protein, 8 aa, synthetic construct
  REGION 1..8: note = Synthetic
GFTFNDYA

SEQ ID NO: 8 - protein, 8 aa, synthetic construct
  REGION 1..8: note = Synthetic
ISWNSDSI

SEQ ID NO: 9 - protein, 20 aa, synthetic construct
  REGION 1..20: note = Synthetic
AKDNHYGSGSYYYYQYGMDV

SEQ ID NO: 10 - protein, 8 aa, synthetic construct
  REGION 1..8: note = Synthetic
GFTFDDYT

SEQ ID NO: 11 - protein, 8 aa, synthetic construct
  REGION 1..8: note = Synthetic
ISWNSGSI

SEQ ID NO: 12 - protein, 16 aa, synthetic construct
  REGION 1..16: note = Synthetic
AKDNSGYGHYYYGMDV

SEQ ID NO: 13 - protein, 6 aa, synthetic construct
  REGION 1..6: note = Synthetic
QSVSSN

SEQ ID NO: 14 - skipped

SEQ ID NO: 15 - protein, 9 aa, synthetic construct
  REGION 1..9: note = Synthetic
QHYINWPLT

SEQ ID NO: 16 - protein, 326 aa, synthetic construct
  REGION 1..326: note = Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 326 aa, synthetic construct
  REGION 1..326: note = Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 18 - protein, 329 aa, synthetic construct
  REGION 1..329: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 329 aa, synthetic construct
  REGION 1..329: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

(19) United States
(12) Patent Application Publication
(10) Pub. No.: US 2025/0137065 A1
(43) Pub. Date: May 1, 2025

(54) Predictive Biomarkers in Patients with Follicular Lymphoma and Diffuse Large B-Cell Lymphoma

(71) Applicant: Regeneron Pharmaceuticals, Inc., Tarrytown, NY (US)

(72) Inventors: Vladimir Jankovic, New York, NY (US); Jurriaan Brouwer, Astoria, NY (US); Nathalie M. Fiaschi, Upper Nyack, NY (US)

(21) Appl. No.: 18/933,903

(22) Filed: Oct. 31, 2024

Related U.S. Application Data
(60) Provisional application No. 63/595,136, filed on Nov. 1, 2023.
(60) Provisional application No. 63/603,989, filed on Nov. 29, 2023.
(60) Provisional application No. 63/652,535, filed on May 28, 2024.

(51) Int. Cl. C12Q 1/6886; C07K 16/28

(52) U.S. Cl. CPC C12Q 1/6886; C07K 16/2809; C07K 16/2887; C07K 2317/31; C12Q 2600/106; C12Q 2600/158

(57) ABSTRACT

The present disclosure provides methods of treating lymphoma comprising administering a bispecific CD20×CD3 antibody to a patient in need thereof, wherein the patient is selected on the basis of exhibiting a modified level of circulating tumor (ct) DNA. In certain embodiments, the present disclosure provides methods of identifying a patient with lymphoma who is likely to respond favorably to therapy comprising a bispecific CD20×CD3 antibody.

### CROSS-REFERENCE TO RELATED APPLICATIONS

This application claims the benefit under 35 USC § 119 (e) of U.S. Provisional Application No. 63/595,136, filed Nov. 1, 2023; 63/603,989, filed Nov. 29, 2023; and 63/652,535, filed May 28, 2024, each of which is incorporated herein by reference in its entirety for all purposes.

### REFERENCE TO A SEQUENCE LISTING

This application incorporates by reference a computer readable Sequence Listing in ST.26 XML format, titled 11667US01_Sequence, created on Oct. 31, 2024 and containing 26,269 bytes.

### FIELD OF THE INVENTION

The present invention lies in the field of medicine, and relates to the use of predictive biomarkers for the selection of patients for the treatment or continued treatment of lymphoma with a bispecific anti-CD3×anti-CD20 antibody.

### BACKGROUND

Molecular characterization of B-cell non-Hodgkin lymphomas to identify various mutations and subtypes is widely used for clinical decision making for patients with lymphoid malignancies (de Leval et al., Blood, 140 (21): 2193-2227, 2022). Although CD20×CD3 bispecific antibodies, including odronextamab, have been demonstrated to produce deep and durable responses in patients with relapsed or refractory lymphomas, specific biomarkers that may predict the efficacy of such therapy or may aid in the identification and selection of patient sub-populations that may respond more favorably to such therapy remain unidentified.

### BRIEF SUMMARY OF THE INVENTION

In one aspect, the present disclosure provides a method of treating lymphoma comprising administering to a subject in need thereof a bispecific antibody comprising a first antigen-binding region that binds human CD20 and a second antigen-binding region that binds human CD3, wherein the subject is selected on the basis of exhibiting a decreased level of circulating tumor (ct) DNA relative to a reference level of ctDNA following an initial period of treatment with the bispecific antibody.

In one aspect, the present disclosure provides a method of selecting a subject for treatment of lymphoma with a bispecific antibody comprising a first antigen-binding region that binds human CD20 and a second antigen-binding region that binds human CD3, the method comprising (a) measuring a level of circulating tumor (ct) DNA in the subject following an initial period of treatment with the bispecific antibody, and (b) comparing the measured level of ctDNA in the subject against a reference level of ctDNA, wherein if the measured level of ctDNA in the subject is less than the reference level of ctDNA, then continuing to administer the bispecific antibody to the subject during a maintenance period.

In various embodiments, the reference level of ctDNA is a baseline level of ctDNA measured prior to the initial period of treatment with the bispecific antibody.

In various embodiments, the initial period of treatment comprises four cycles of treatment, wherein each cycle comprises weekly administration of a dose of the bispecific antibody. In some embodiments, each cycle is three weeks in duration. In some cases, the weekly administration of the dose during cycle 1 comprises step-up dosing, and/or split dosing wherein two dose fractions of the dose are administered on consecutive days.

In various embodiments, the decreased level of ctDNA corresponds to minimal residual disease (MRD) negativity.

In various embodiments, the lymphoma is follicular lymphoma or diffuse large B-cell lymphoma.

In various embodiments, treating lymphoma comprises further administering the bispecific antibody during a maintenance period. In some cases, the bispecific antibody is administered once every other week during the maintenance period.

In one aspect, the present disclosure provides a method of treating lymphoma comprising administering to a subject in need thereof a bispecific antibody comprising a first antigen-binding region that binds human CD20 and a second antigen-binding region that binds human CD3, wherein the subject is selected on the basis of exhibiting an absence of tumor protein p53 mutations, as measured by circulating tumor (ct) DNA. In some cases, the lymphoma is follicular lymphoma or diffuse large B-cell lymphoma.

In one aspect, the present disclosure provides a method of treating diffuse large B-cell lymphoma (DLBCL) comprising administering to a subject in need thereof a bispecific antibody comprising a first antigen-binding region that binds human CD20 and a second antigen-binding region that binds human CD3, wherein the subject is selected on the basis of exhibiting genetic modifications corresponding to an EZB subtype, as measured by circulating tumor (ct) DNA. In some cases, the DLBCL is a germinal-center B-cell-like subtype. In some cases, the DLBCL is a non-germinal-center B-cell-like subtype.

In various embodiments, the subject has relapsed or refractory disease.

In various embodiments, the first antigen-binding region of the bispecific antibody comprises three heavy chain complementarity determining regions, HCDR1, HCDR2 and HCDR3 comprising the amino acid sequences of SEQ ID NO: 7, 8 and 9, respectively, and three light chain complementarity determining regions LCDR1, LCDR2 and LCDR3 comprising the amino acid sequences of SEQ ID NO: 13, 14 and 15, respectively, and wherein the second antigen-binding region of the bispecific antibody comprises three heavy chain complementarity determining regions, HCDR1, HCDR2 and HCDR3 comprising the amino acid sequences of SEQ ID NO: 10, 11 and 12, respectively, and three light chain complementarity determining regions LCDR1, LCDR2 and LCDR3 comprising the amino acid sequences of SEQ ID NO: 13, 14 and 15, respectively. In some cases, the first antigen-binding region of the bispecific antibody comprises a heavy chain variable region (HCVR) comprising the amino acid sequence of SEQ ID NO: 4 and a light chain variable region (LCVR) comprising the amino acid sequence of SEQ ID NO: 6, and the second antigen-binding region of the bispecific antibody comprises a HCVR comprising the amino acid sequence of SEQ ID NO: 5 and a LCVR comprising the amino acid sequence of SEQ ID NO: 6. In some cases, the bispecific antibody comprises a human IgG heavy chain constant region, optionally of isotype IgG1 or IgG4. In some embodiments, the bispecific antibody comprises a first heavy chain comprising amino acid residues 1-452 of SEQ ID NO: 1 paired with a common light chain comprising the amino acid sequence of SEQ ID NO: 3, and a second heavy chain comprising amino acid residues 1-448 of SEQ ID NO: 2 paired with a common light chain comprising the amino acid sequence of SEQ ID NO: 3. In some embodiments, the bispecific antibody comprises a first heavy chain comprising the amino acid sequence of SEQ ID NO: 1 paired with a common light chain comprising the amino acid sequence of SEQ ID NO: 3, and a second heavy chain comprising the amino acid sequence of SEQ ID NO: 2 paired with a common light chain comprising the amino acid sequence of SEQ ID NO: 3. In some cases, the bispecific antibody is odronextamab.

In various embodiments of the methods: (a) the subject has been diagnosed with follicular lymphoma; (b) the subject has been diagnosed with follicular lymphoma of grade 1-3a; (c) the subject has been diagnosed with relapsed or refractory follicular lymphoma after at least 2 prior lines of systemic therapy; (d) the subject has been diagnosed with follicular lymphoma and has not previously been treated with a systemic anti-lymphoma therapy; (e) the subject has been diagnosed with follicular lymphoma, and the full dose is 80 mg; and/or (f) the subject has been diagnosed with follicular lymphoma, and the maintenance dose is 160 mg or 320 mg.

In various embodiments of the methods: (a) the subject has been diagnosed with diffuse large B-cell lymphoma (DLBCL); (b) the subject has been diagnosed with DLBCL, and wherein the DLBCL is de novo or is transformed from a lower grade neoplasm; (c) the subject has been diagnosed with DLBCL and is refractory to at least 2 prior lines of systemic therapy; (d) the subject has been diagnosed with relapsed or refractory DLBCL after at least 2 prior lines of systemic therapy including CAR-T therapy; (e) the subject has been diagnosed with DLBCL and has not previously been treated with a systemic anti-lymphoma therapy; (f) the subject has been diagnosed with DLBCL, and the full dose is 160 mg; and/or (g) the subject has been diagnosed with DLBCL, and the maintenance dose is 320 mg.

In various embodiments of the methods, the subject has a detectable level of CD20 in a tumor cell sample prior to treatment with the bispecific antibody, as measured by mRNA expression and/or immunohistochemistry. In other embodiments of the methods, subject has a non-detectable level of CD20 in a tumor cell sample prior to treatment with the bispecific antibody, as measured by mRNA expression and/or immunohistochemistry.

In one aspect, the present disclosure provides methods of treating lymphoma comprising administering a bispecific CD20×CD3 antibody to a patient in need thereof, wherein the patient is predicted to respond to the therapy, and wherein the patient exhibits a level of circulating tumor DNA (ct DNA) that is indicative of minimal residual disease negativity.

In one aspect, the present disclosure provides methods of treating lymphoma comprising administering a bispecific CD20×CD3 antibody to a patient in need thereof, wherein the patient is predicted to respond to the therapy, and wherein the patient does not exhibit TP53 mutations.

The present disclosure also contemplates the use of an anti-CD20×anti-CD3 antibody (e.g., odronextamab) for use in a method as discussed above or herein, as well as use of an anti-CD20×anti-CD3 antibody (e.g., odronextamab) in the manufacture of a medicament for treating a B-cell cancer in a subject (e.g., FL or DLBCL) according to a method as discussed above or herein.

In various embodiments, any of the features or components of any embodiments discussed above or herein may be combined, and such combinations are encompassed within the scope of the present disclosure. Any specific value discussed above or herein may be combined with another related value discussed above or herein to recite a range with the values representing the upper and lower ends of the range, and such ranges are encompassed within the scope of the present disclosure. A therapeutic protein for use in any of the methods discussed herein, or use of a therapeutic protein in the manufacture of a medicament for use in any of the methods discussed herein are also encompassed within the scope of this disclosure.

Other embodiments will become apparent from a review of the ensuing detailed description.

### BRIEF DESCRIPTION OF THE DRAWINGS

FIGS. 1A and 1B illustrate survival probability in follicular lymphoma (FL) (FIG. 1A) and diffuse large B-cell lymphoma (DLBCL) (FIG. 1B) patients with circulating tumor (ct) DNA detected following 4 cycles of treatment with odronextamab vs. patients without ctDNA detected following 4 cycles of treatment with odronextamab, as discussed in Example 1. As shown in the K-M curves, the median progression free survival (PFS) was longer in patients who were minimal residual disease (MRD) negative at cycle 4, day 15 (C4D15) compared with patients who still had detectable MRD at that timepoint, in both FL and DLBCL. The hazard ratios were 0.26 and 0.34, respectively.

FIGS. 2A, 2B, 2C and 2D illustrate survival probability in FL patients (FIGS. 2A and 2B) and DLBCL patients (FIGS. 2C and 2D) based on a combination of MRD with PET-CT complete response (CR) status, as discussed in Example 1. Among patients with FL, MRD status was predictive of PFS benefit, even in patients who achieved a CR at C4D15 (FIG. 2A), with a hazard ratio of 0.30. In patients with DLBCL who did not achieve a CR at C4D15, MRD status was predictive of PFS (FIG. 2D), with a hazard ratio of 0.25.

FIGS. 3A and 3B illustrate survival probability as a function of baseline TP53 mutations in ctDNA, as discussed in Example 1. TP53 was the most frequent mutation observed in evaluable patients at baseline, and was indicative of reduced PFS in FL and DLBCL patients.

FIG. 4 illustrates survival probability in DLBCL patients classified as MCD subtype vs. EZB subtype vs. other subtypes, as determined by ctDNA, as discussed in Example 1. Lymphgen classification was applied to 84 patients with DLBCL. The most prevalent subtypes were MCD and EZB. Median PFS was longer among EZB versus MCD patients. “Other” patients included those with ST2 or other LymphGen classification. EZB, including EZH2 mutations and BCL2 translocations; MCD, including MYD88L265P and CD79B mutations; ST2, SGK1 and TET2 mutated.

FIG. 5 illustrates survival probability as a function of baseline cell of origin in DLBCL patients, as discussed in Example 1. Cell of origin is a known prognostic factor in DLBCL and was evaluable in 85 patients. As shown in FIG. 5, median PFS was improved in the subgroup of patients with germinal center B-like (or GCB) disease compared with the non-GCB subtype.

FIGS. 6A and 6B illustrate the relationship between PFS and CD20 expression in FL patients, as discussed in Example 2.

FIGS. 7A and 7B illustrate the relationship between duration of response (DOR) and CD20 expression in FL patients, as discussed in Example 2.

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. The term “about,” when used in reference to a particular recited numerical value, means that the value may vary from the recited value by no more than 1%. For example, the expression “about 100” includes 99 and 101 and all values in between (e.g., 99.1, 99.2, 99.3, 99.4, etc.).

Although any methods and materials similar or equivalent to those described herein can be used in the practice or testing of the present invention, the preferred methods and materials are now described. All patents, applications and non-patent publications mentioned in this specification are incorporated herein by reference in their entireties.

### Definitions

All references to proteins, polypeptides and protein fragments herein are intended to refer to the human version of the respective protein, polypeptide or protein fragment unless explicitly specified as being from a non-human species. Thus, the expressions “CD3” and “CD20” mean human CD3 and human CD20, respectively, unless specified as being from a non-human species.

The expression “CD3,” refers to an antigen which is expressed on T cells as part of the multimolecular T cell receptor (TCR) and which consists of a homodimer or heterodimer formed from the association of two of four receptor chains: CD3-epsilon, CD3-delta, CD3-zeta, and CD3-gamma.

“An antigen-binding domain that binds CD3,” “an antigen-binding region that binds CD3,” “an antibody that binds CD3” or an “anti-CD3 antibody” includes antibodies and antigen-binding fragments thereof that specifically recognize a single CD3 subunit (e.g., epsilon, delta, gamma or zeta), as well as antibodies and antigen-binding fragments thereof that specifically recognize a dimeric complex of two CD3 subunits (e.g., gamma/epsilon, delta/epsilon, and zeta/zeta CD3 dimers). The antibodies and antigen-binding fragments of the present invention may bind soluble CD3 and/or cell surface expressed CD3. Soluble CD3 includes natural CD3 proteins as well as recombinant CD3 protein variants such as, e.g., monomeric and dimeric CD3 constructs, that lack a transmembrane domain or are otherwise unassociated with a cell membrane.

The expression “CD20,” refers to a non-glycosylated phosphoprotein expressed on the cell membranes of mature B cells. CD20 is considered a B cell tumor-associated antigen because it is expressed by more than 95% of B-cell non-Hodgkin lymphomas (NHLs) and other B-cell malignancies, but it is absent on precursor B-cells, dendritic cells and plasma cells.

“An antigen-binding domain that binds CD20,” “an antigen-binding region that binds CD20,” “an antibody that binds CD20” or an “anti-CD20 antibody” includes antibodies and antigen-binding fragments thereof that specifically recognize CD20.

Non-Hodgkin lymphomas can be divided into two major prognostic groups: indolent (low-grade; slowly growing) lymphomas, and aggressive (high-grade; quickly growing) lymphomas. An “aggressive lymphoma” is a lymphoma characterized by one of the following subtypes based on the World Health Organization classification: a diffuse large B-cell lymphoma (DLBCL) not otherwise specified (NOS) by WHO classification; germinal center B-cell type; activated B-cell type; primary mediastinal (thymic) large B-cell lymphoma; T-cell/histiocyte-rich large B-cell lymphoma; Epstein-Barr virus (EBV)+ DLBCL, NOS; high-grade B-cell lymphoma, with MYC and BCL2 and/or BCL6 rearrangements; high-grade B-cell lymphoma, NOS; B-cell lymphoma, unclassifiable, with features intermediate between DLBCL and classical Hodgkin lymphoma; and follicular lymphoma, grade 3b. Follicular lymphoma, grade 1-3a, is the most common form of indolent lymphoma.

The term “antibody”, means any antigen-binding molecule or molecular complex comprising at least one complementarity determining region (CDR) that specifically binds to or interacts with a particular antigen (e.g., CD20 or CD3). The term “antibody” includes immunoglobulin molecules comprising four polypeptide chains, two heavy (H) chains and two light (L) chains inter-connected by disulfide bonds, as well as multimers thereof (e.g., IgM). The term “antibody” also includes immunoglobulin molecules consisting of four polypeptide chains, two heavy (H) chains and two light (L) chains inter-connected by disulfide bonds. Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs of the antibody (or antigen-binding portion thereof) may be identical to the human germline sequences, or may be naturally or artificially modified. An amino acid consensus sequence may be defined based on a side-by-side analysis of two or more CDRs. The term “antibody” includes a “bispecific antibody” unless otherwise noted. Antibodies of the present disclosure may include a human IgG heavy chain. In various embodiments, the heavy chain constant region may be of IgG1, IgG2, IgG3 or IgG4 isotype. In some cases, the heavy chain constant region is of isotype IgG1. In some cases, the heavy chain constant region is of isotype IgG4. In certain embodiments, the antibodies or bispecific antibodies are human antibodies. The term “human antibody” is intended to include antibodies having variable and constant regions derived from human germline immunoglobulin sequences.

The antibodies of the invention may, in some embodiments, be recombinant human antibodies. The term “recombinant human antibody” is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

### Biomarkers Predictive of Therapeutic Efficacy in the Treatment of Lymphoma

The present disclosure includes the identification of biomarkers that are predictive of therapeutic efficacy in the treatment of lymphoma. In some cases, the patient being evaluated has follicular lymphoma (FL) or diffuse large B-cell lymphoma (DLBCL). In embodiments, the biomarkers may be a measured level of circulating tumor (ct) DNA that may be indicative of minimal residual disease (MRD) negativity, or may be specific mutations identified within ctDNA (e.g., mutations in tumor protein p53), or may be genetic modifications corresponding to a specific subtype of lymphoma (e.g., the EZB subtype of DLBCL) identified within ctDNA. The use of ctDNA to identify biomarkers predictive of therapeutic efficacy provides a non-invasive method for molecular characterization of lymphoma patients with no available tissue, and can be used to select patients for treatment, or continued treatment, with an anti-CD20×CD3 bispecific antibody. ctDNA measurements may be performed as discussed in, for example, Newman et al., Nat. Med., 20:548-554, 2014; Newman et al., Nat. Biotechnol., 34:547-555, 2016; or Kurtz et al., Journal of Clinical Oncology, 36 (28): 2845-2853, 2018.

In some cases, the ctDNA is measured at baseline, i.e., before the patient has been treated with a CD20×CD3 bispecific antibody (as discussed herein). In some cases, the ctDNA is measured following one or more cycles (e.g., after four 21-day cycles) of treatment with a CD20×CD3 bispecific antibody to gauge the patient's early response to therapy. In embodiments, the measured level of ctDNA may be compared to a reference level of ctDNA, which may be a baseline measurement (e.g., if the measured level follows one or more cycles of treatment) or a measurement taken at an earlier period within a treatment regimen. In some embodiments, the reference level may be a level representative of a population of healthy individuals, or a level known to be associated with MRD negativity. As demonstrated in the examples, below, FL and DLBCL patients with ctDNA levels associated with MRD negativity after four cycles (each cycle is three weeks with weekly dosing) are shown to have significantly longer progression-free survival when treated with an anti-CD20×CD3 bispecific antibody (e.g., odronextamab).

In various embodiments, the reference level of ctDNA is a baseline level of ctDNA measured prior to the initial period of treatment with the bispecific antibody. In some cases, the ctDNA is measured following the initial period of treatment, which may comprise one or more cycles of treatment. The initial period of treatment may comprise 1, 2, 3, 4, 5, 6, 7, 8, 9 or 10 or more (e.g., 11, 12, 13, 14, 15, 16, 17, 18, 19 or 20) cycles of treatment, wherein each cycle may be 1-7 days in duration, 1 week, 2 weeks, 3 weeks, 4 weeks, 5 weeks, 6, weeks, 7, weeks, or 8 weeks or more in duration. In some embodiments, the initial period of treatment is one cycle (e.g., three or four weeks in duration). In some embodiments, the initial period of treatment is two cycles (e.g., three or four weeks in duration). In some embodiments, the initial period of treatment is three cycles (e.g., three or four weeks in duration). In some embodiments, the initial period of treatment is four cycles (e.g., three or four weeks in duration). In some embodiments, the initial period of treatment is two, three or four cycles (e.g., each three weeks in duration). During each cycle, the bispecific antibody may be administered daily, every other day, every three days, weekly, twice weekly, three times weekly, four times weekly, every other week, every three weeks, or the like. In some embodiments, the bispecific antibody is administered weekly.

In some cases, the ctDNA is measured to identify specific mutations or modifications present in the tumor DNA to determine whether the individual patient has a mutation associated with an increased or decreased probability of therapeutic efficacy, or whether the individual patient has a mutation or mutations associated with a particular subtype of lymphoma (e.g., via LymphGen classification) associated with an increased or decreased probability of therapeutic efficacy. In some cases, the ctDNA is measured at baseline, and/or during treatment to determine whether a patient has a tumor protein p53 mutation. In some cases, the ctDNA is measured at baseline, and/or during treatment to determine whether the patient has genetic modifications associated with the EZB subtype of DLBCL. As demonstrated in the examples, below, FL and DLBCL patients without tumor protein p53 mutations, DLBCL patients with the EZB subtype, DLBCL patients with germinal-center B-cell like (GCB) cell of origin, and FL patients with CD20+ tumors (by mRNA or IHC; e.g., with ≥10% CD20+ cells, an H-score of ≥50, or a median mRNA CD20 TPM (transcripts per million) of ≥1664.8) are shown to have significantly longer progression-free survival when treated with an anti-CD20×CD3 bispecific antibody (e.g., odronextamab).

### Therapeutic Uses of the Bispecific Antibodies

The present invention includes methods of treating lymphomas in a subject in need thereof selected on the basis of the predictive biomarkers discussed above. The bispecific antibodies may be contained in a composition comprising a pharmaceutically acceptable carrier or diluent. The expressions “a subject” or “a subject in need thereof” mean a human or non-human animal that exhibits one or more symptoms or indicia of cancer (e.g., a subject expressing a tumor or suffering from any of the cancers mentioned herein below).

In some embodiments, the bispecific anti-CD3×anti-CD20 antibodies are useful for treating a B-cell malignancy including non-Hodgkin lymphoma, Hodgkin lymphoma, chronic lymphocytic leukemia, acute lymphoblastic leukemia, small lymphocytic lymphoma, diffuse large B-cell lymphoma, follicular lymphoma, mantle cell lymphoma, marginal zone lymphoma, Waldenstrom macroglobulinemia, primary mediastinal B-cell lymphoma, lymphoblastic lymphoma, or Burkitt lymphoma. In some embodiments, the cancer is follicular lymphoma. In some embodiments, the cancer is diffuse large B-cell lymphoma (DLBCL). In some embodiments, the cancer is mantle cell lymphoma. In some embodiments, the cancer is marginal zone lymphoma.

Non-Hodgkin Lymphoma (NHL) is the most common hematological malignancy. Among a heterogeneous group of NHLs, 85-90% are of B-cell origin and include follicular lymphoma (FL), diffuse large B-cell lymphoma (DLBCL), mantle cell lymphoma (MCL), marginal zone lymphoma (MZL), and several other B-NHLs. Anti-CD20 antibodies in combination with chemotherapy are the standard of care for the treatment of B-NHLs; however, despite initial responses, many patients relapse, often with progressively shorter response durations in subsequent lines of therapy and poor outcomes. Thus, in some embodiments, the antigen-binding molecule is a bispecific anti-CD3×anti-CD20 that binds to CD3₊ T cells and CD20₊ B cells, targeting CD20₊ tumor cells via T-cell mediated cytotoxicity. In some cases, the anti-CD3×CD20 bispecific antibody is for treatment of a B-cell cancer (e.g., a NHL) in a subject that has failed prior therapy with an anti-CD20 monospecific antibody.

For patients with less than a complete response to CAR-T therapy, the outcomes are generally poor, and there are no standard-of-care therapeutic options. Thus, in some cases, the anti-CD3×CD20 bispecific antibody of the present invention is for treatment of a B-cell cancer (e.g., a NHL such as DLBCL) in a subject that has failed prior CAR-T therapy or is not responsive to prior CAR-T therapy (e.g., anti-CD19 CAR-T therapy).

In an embodiment, odronextamab is indicated for the treatment of adult patients with relapsed or refractory follicular lymphoma (FL) after at least two prior systemic therapies.

In an embodiment, odronextamab is indicated for the treatment of adult patients diagnosed with follicular lymphoma or DLBCL who have not been previously treated with any systemic anti-lymphoma therapy.

For relapsed or refractory follicular lymphoma (R/R FL), administration may be via an intravenous (IV) infusion. Treatment consists of step-up dosing in cycle 1, weekly dosing in cycles 2-4 followed by maintenance dosing every 2 weeks until disease progression, as for example, shown in Table 1, below.

A single treatment cycle (for FL) consists of 21 days.

Cycle 1: Step Up-Administer odronextamab as a 4 hour infusion. The recommended starting dose of odronextamab is 0.2 mg on day 1. If tolerated, administer 0.5 mg on day 2. If tolerated, administer a dose of 2 mg on day 8 and 2 mg on day 9. If tolerated, administer a dose of 10 mg on day 15 and 10 mg on day 16. If tolerated, administer cycle 2.
Cycles 2-4:80 mg Weekly-Administer 80 mg as a 4 hour infusion on cycle 2, day 1. If tolerated, infusion time can be reduced to 1 hour for all subsequent doses. Administer a dose of 80 mg on day 1, day 8 and day 15.
Maintenance: 160 mg Every 2 Weeks-After cycle 4, administer odronextamab at a dose of 160 mg as a 1 hour infusion every two weeks. If a patient is in complete remission for 9 months, administer a dose of 160 mg every 4 weeks, or 320 mg every 8 weeks.

TABLE 1
Odronextamab Dose and Schedule for the Treatment of R/R FL

| | | Dose of IV |
|---|---|---|
| Day of Treatment Cycle | | Odronextamab (mg) |
| Cycle 1 | Day 1 | 0.2 |
| (Step Up) | Day 2^{a} | 0.5 |
| | Day 8 | 2 |
| | Day 9^{b} | 2 |
| | Day 15 | 10 |
| | Day 16^{c} | 10 |
| Cycles 2-4 | Day 1 | 80 |
| | Day 8 | 80 |
| | Day 15 | 80 |
| Maintenance | Begin 2 weeks after Cycle 4, | 160 |
| (Every 2 Weeks) | Day 15. | |

^{a}Dose on Day 2 can administered on Day 2, 3 or 4.
^{b}Dose on Day 9 can administered on Day 9, 10 or 11.
^{c}Dose on Day 16 can administered on Day 16, 17 or 18.

In an embodiment, odronextamab is indicated for the treatment of adult patients with relapsed or refractory diffuse large B-cell lymphoma (DLBCL) after at least two prior systemic therapies.

For diffuse large B-cell lymphoma (DLBCL), administration may be via an intravenous (IV) infusion. Treatment consists of step-up dosing in cycle 1, weekly dosing in cycles 2-4 followed by maintenance dosing every 2 weeks until disease progression, as for example, shown in Table 2, below.

A single treatment cycle (for DLBCL) consists of 21 days.

Cycle 1: Step Up-Administer odronextamab as a 4 hour infusion. The recommended starting dose of odronextamab is 0.2 mg on day 1. If tolerated, administer 0.5 mg on day 2. If tolerated, administer a dose of 2 mg on day 8 and 2 mg on day 9. If tolerated, administer a dose of 10 mg on day 15 and 10 mg on day 16. If tolerated, administer cycle 2.
Cycles 2-4:160 mg Weekly-Administer 160 mg as a 4 hour infusion on cycle 2, day 1. If tolerated, infusion time can be reduced to 1 hour for all subsequent doses. Administer a dose of 160 mg on day 1, day 8 and day 15.
Maintenance: 320 mg Every 2 Weeks-After cycle 4, administer odronextamab at a dose of 320 mg as a 1 hour infusion every two weeks. If a patient is in complete remission for 9 months, administer a dose of 320 mg every 4 weeks.

TABLE 2
Odronextamab Dose and Schedule
for the Treatment of R/R DLBCL

| | | Dose of IV |
|---|---|---|
| Day of Treatment Cycle | | Odronextamab (mg) |
| Cycle 1 | Day 1 | 0.2 |
| (Step Up) | Day 2^{a} | 0.5 |
| | Day 8 | 2 |
| | Day 9^{b} | 2 |
| | Day 15 | 10 |
| | Day 16^{c} | 10 |
| Cycles 2-4 | Day 1 | 160 |
| | Day 8 | 160 |
| | Day 15 | 160 |
| Maintenance | Begin 2 weeks after Cycle 4, | 320 |
| (Every 2 Weeks) | Day 15. | |

^{a}Dose on Day 2 can administered on Day 2, 3 or 4.
^{b}Dose on Day 9 can administered on Day 9, 10 or 11.
^{c}Dose on Day 16 can administered on Day 16, 17 or 18.

A summary of the sequences and the corresponding SEQ ID NOs referenced herein is shown in Table 3, below. The anti-CD3×anti-CD20 antibody comprising the heavy chains and common light chain of SEQ ID NOs: 1-3, the HCVRs and LCVR of SEQ ID NOs: 4-6, and the CDRs of SEQ ID NOs: 7-15, is also referred to herein as odronextamab.

TABLE 3
Summary of Sequences

| SEQ ID NO: | Description |
|---|---|
| 1 | Anti-CD20 Heavy Chain |
| 2 | Anti-CD3 Heavy Chain |
| 3 | Common Anti-CD20 and Anti-CD3 Light Chain |
| 4 | Anti-CD20 HCVR |
| 5 | Anti-CD3 HCVR |
| 6 | Common Anti-CD20 and Anti-CD3 LCVR |
| 7 | Anti-CD20 HCDR1 |
| 8 | Anti-CD20 HCDR2 |
| 9 | Anti-CD20 HCDR3 |
| 10 | Anti-CD3 HCDR1 |
| 11 | Anti-CD3 HCDR2 |
| 12 | Anti-CD3 HCDR3 |
| 13 | Common Anti-CD20 and Anti-CD3 LCDR1 |
| 14 | Common Anti-CD20 and Anti-CD3 LCDR2 |
| 15 | Common Anti-CD20 and Anti-CD3 LCDR3 |
| 16 | IgG4 Heavy Chain Constant Region |
| 17 | IgG4 Heavy Chain Constant Region with H435R/Y436F |
| 18 | IgG1 Heavy Chain Constant Region |
| 19 | IgG1 Heavy Chain Constant Region with H435R/Y436F |

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Circulating Tumor DNA (ctDNA) Analysis Predicts Progression-Free Survival (PFS) with Odronextamab Monotherapy in Relapsed/Refractory (R/R) Follicular Lymphoma (FL) and Diffuse Large B-Cell Lymphoma (DLBCL): Identification of Minimal Residual Disease (MRD) Status and High-Risk Subgroups

Background: Molecular characterization of B-cell non-Hodgkin lymphoma (B-NHL), through ctDNA MRD assessment or subtype classification, may be used as a tool to predict clinical outcome. Odronextamab, a CD20×CD3 bispecific antibody, demonstrated deep and durable responses in patients with R/R FL (ORR of 80.5%) or DLBCL (ORR of 52%) in the Phase 2 ELM-2 study (NCT03888105; Kim T M et al. and Kim W S et al. ASH. 2022). In the overall populations, PFS rates at 12 months were 64% and 29%, respectively. Using tumor biopsy and ctDNA from ELM-2, this example demonstrates that these assessments can predict response to odronextamab treatment.

Methods: In ELM-2, patients received IV odronextamab in 21-day cycles, with step-up dosing in Cycle (C) 1, 80 mg (FL)/160 mg (DLBCL) QW in C2-4, then 160 mg (FL)/320 mg (DLBCL) Q2W until disease progression or unacceptable toxicity. Baseline (BL) ctDNA and tumor biopsies were used for molecular profiling. BL and on-treatment ctDNA were used for MRD determination in the biomarker population (BP; patients required ≥1 available plasma biomarker sample to be included in the BP). The first post-BL ctDNA sample was collected on C4 Day (D) 15, at the time of positron emission tomography-computed tomography (PET-CT). A modified AVENIO ctDNA analysis workflow and pipeline (Roche; research only) was used for next-generation sequencing, based on the cancer personalized profiling by deep sequencing technique (Kurtz et al. J Clin Oncol. 2018). Whole blood cell pellets were used to filter out germline allele variants. MRD negativity was reported when the P value for variant allele frequency was >0.005.

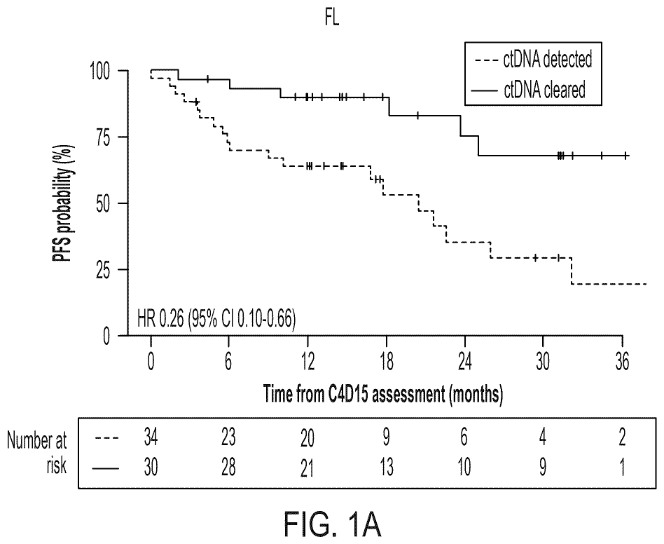

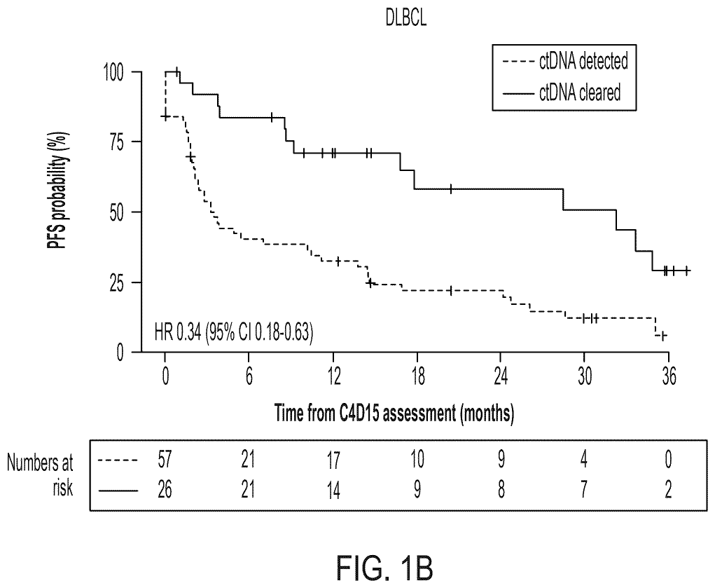

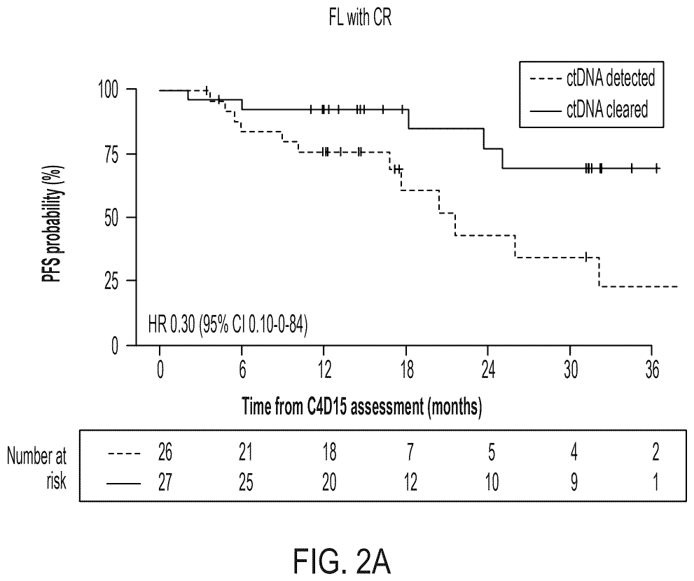

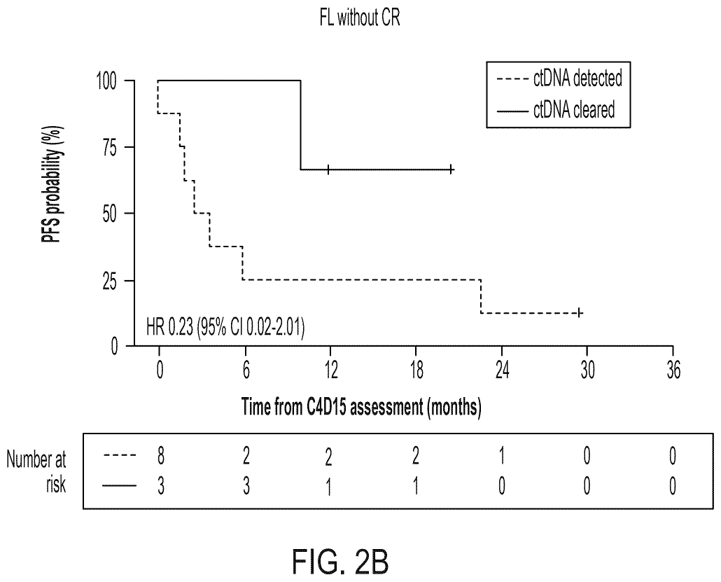

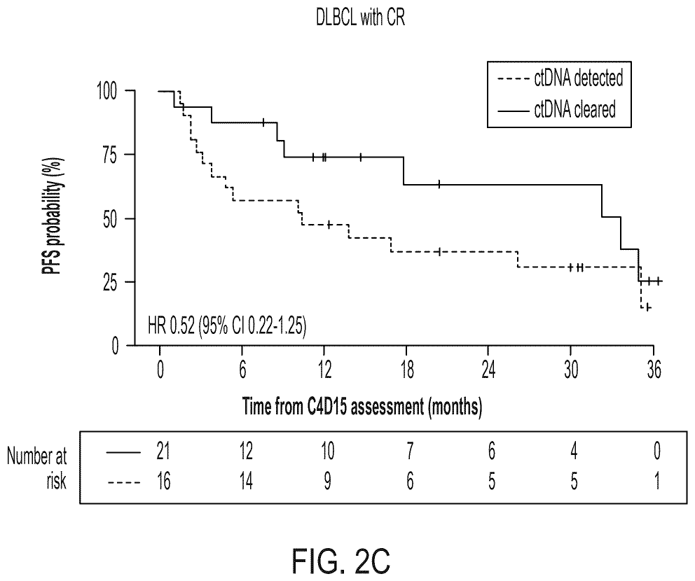

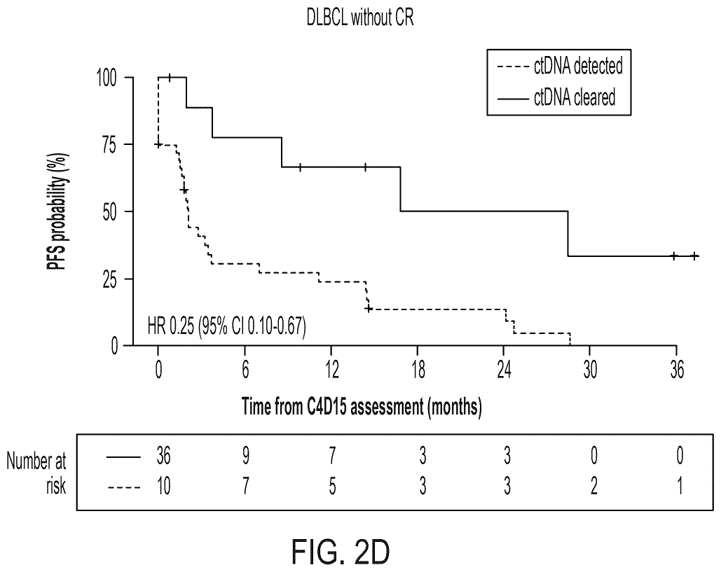

Results: The overall population and the BP had similar baseline characteristics. The BP comprised 70 FL patients and 93 DLBCL patients; at BL, 64 FL patients and 83 DLBCL patients were MRD (+). Patients remaining on study until C4D15 had similar PFS regardless of whether they were in the BP or overall population (FL, n=128; DLBCL, n=160). Patients who were MRD (−) at C4D15 had significantly longer PFS vs. those who remained MRD (+) (FL: HR 0.26 [95% CI 0.10-0.66], [FIG. 1A]; DLBCL: HR 0.34 [95% CI 0.18-0.63], [FIG. 1B]). As shown in FIGS. 2A-2D, MRD status predicted PFS benefit in FL patients who achieved a CR at C4D15 [FIG. 2A]. and MRD status predicted PFS benefit in DLBCL patients who did not achieve a CR at C4D15 [FIG. 2D].

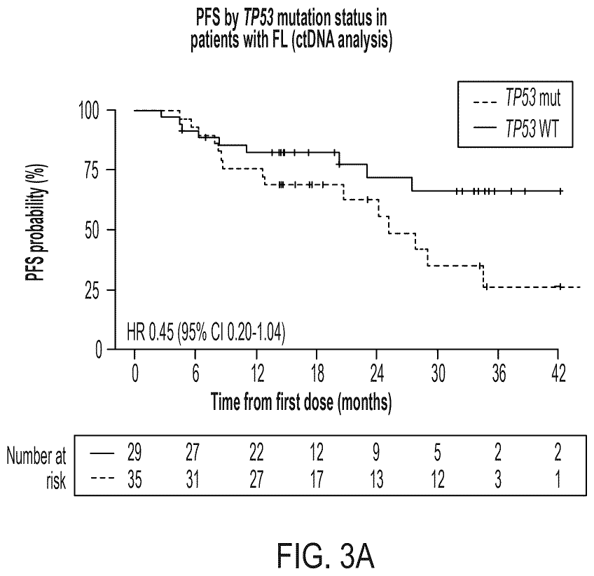

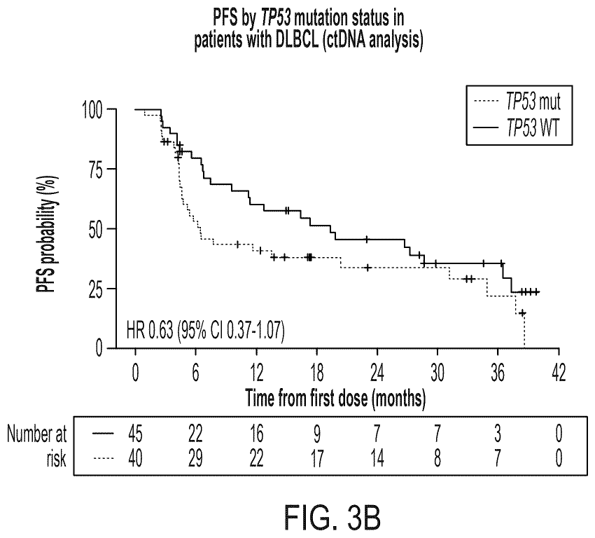

Mutational analyses of BL ctDNA identified TP53 as the most frequent mutation in both FL and DLBCL (FL, n=29/64 [45%]; DLBCL, n=45/85 [53%]). TP53 mutations were indicative of reduced PFS in FL and DLBCL patients, as shown in FIGS. 3A and 3B.

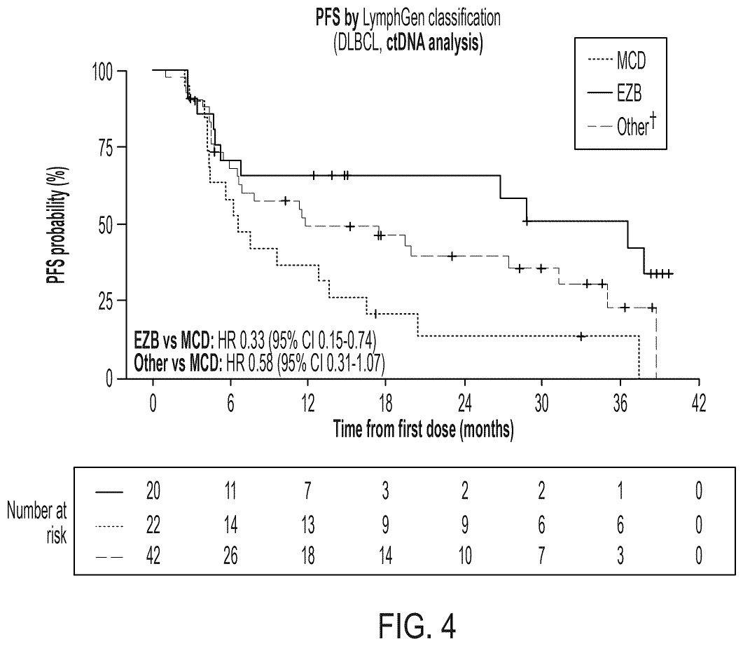

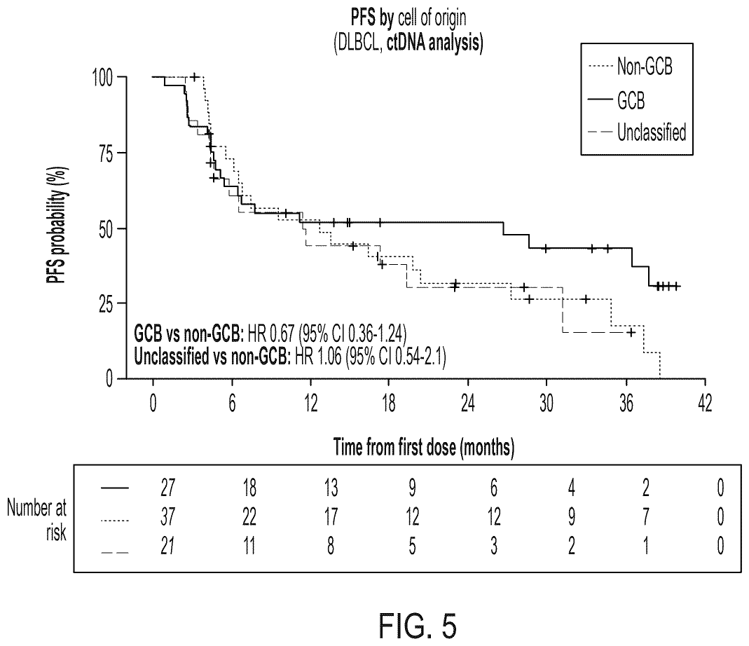

LymphGen classification (Wright et al. Cancer Cell. 2023) of BL ctDNA in patients with DLBCL identified mostly MCD and EZB subtypes (MCD, n=20; EZB, n=22; ST2, n=5; other, n=37). EZB-subtype patients treated with odronextamab had significantly longer PFS compared with MCD-subtype patients, as shown in FIG. 4. Using BL ctDNA, cell of origin was determined in 85 patients with DLBCL. Germinal center B-cell like (GCB) cell of origin was associated with improved PFS versus non-GCB, as shown in FIG. 5 . . . . Further molecular assessment focused on gene fusions in DLBCL patients in the BP.

Conclusions: The study presented in this example is among the first to prospectively analyze ctDNA in patients with R/R FL and DLBCL in the setting of a pivotal trial. This non-invasive method allows molecular characterization of patients with no available tissue, enabling identification of high-risk subgroups. CtDNA MRD status at C5D1 of odronextamab treatment was highly predictive of PFS in patients with FL and DLBCL and could form the basis of response-directed treatment paradigms.

### Example 2: Analyses of the Association of CD20 Expression with Clinical Outcomes in Follicular Lymphoma (FL) and Diffuse Large B-Cell Lymphoma (DLBCL) Patients

Background: CD20 is a well-validated treatment target for B-NHL, yet the impact of CD20 expression level on efficacy of bispecific antibodies (BsAbs) is poorly understood. Odronextamab, a CD20×CD3 BsAb, demonstrated deep and durable responses and generally manageable safety in patients (pts) with R/R follicular lymphoma (FL) and diffuse large B-cell lymphoma (DLBCL) in the ELM-2 study (Villasboas, et al. & Ayyappan, et al. ASH 2023). This example reports post-hoc analyses of the association of CD20 expression with clinical outcomes in FL and DLBCL patients from ELM-2.

Methods: ELM-2 enrolled adult patients with B-NHL who were R/R after ≥2 prior lines of therapy. IV odronextamab was given in 21-day cycles until disease progression/unacceptable toxicity, as discussed in Example 1. Primary endpoint was objective response rate (ORR). Baseline biopsies or archival tissue were evaluated for CD20 expression by mRNA level and immunohistochemistry (IHC). Fully automated chromogenic IHC was performed centrally, and H-score (McCarty, et al. Arch Pathol Lab Med. 1985) calculated. CD20 expression was classified into tertiles and relationship with outcomes determined.

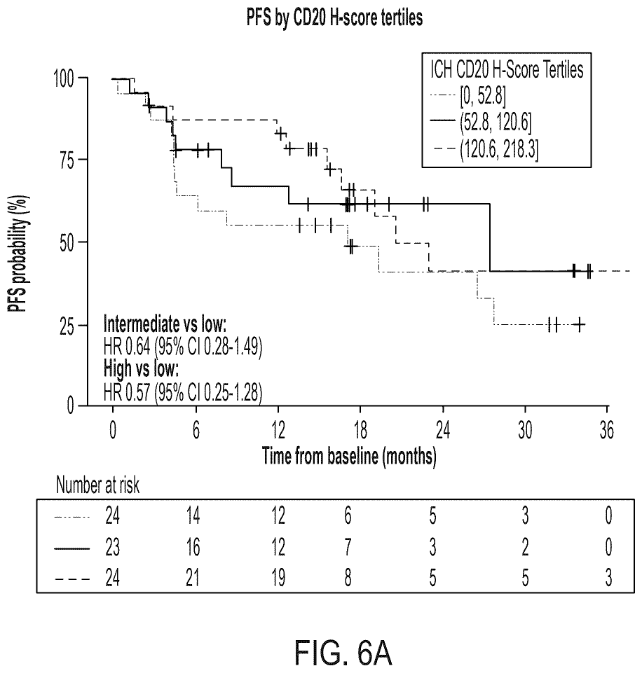

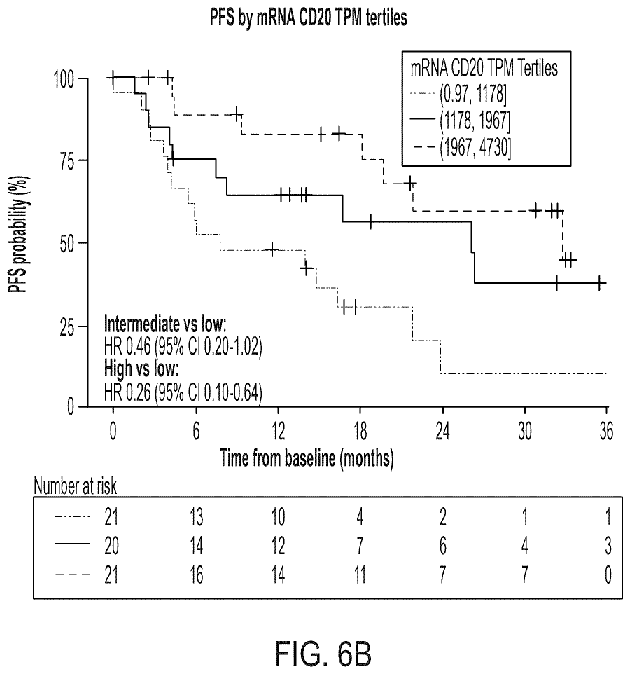

Results: At baseline, tissue was available from 127 pts (FL n=62; DLBCL n=65) for mRNA and 111 (FL n=71; DLBCL n=40) for IHC. Median sample age at baseline was 25 days. Baseline CD20 expression by IHC and mRNA analysis was higher in patients with FL than DLBCL, but responses were even achieved in some CD20-patients. Tertile analysis in FL patients showed that higher CD20 H-score and higher CD20 mRNA expression were associated with longer PFS. In particular, the PFS benefit with high versus low tertiles of CD20 expression was most prominent with mRNA analysis. A PFS benefit was observed in FL patients with ≥10% CD20+ cells (predefined), an H-score of ≥50 (predefined), or a median mRNA CD20 TPM (transcripts per million) of ≥1664.8 (FIGS. 6A and 6B). PFS was not associated with CD20 expression in patients with DLBCL, regardless of IHC or mRNA analysis.

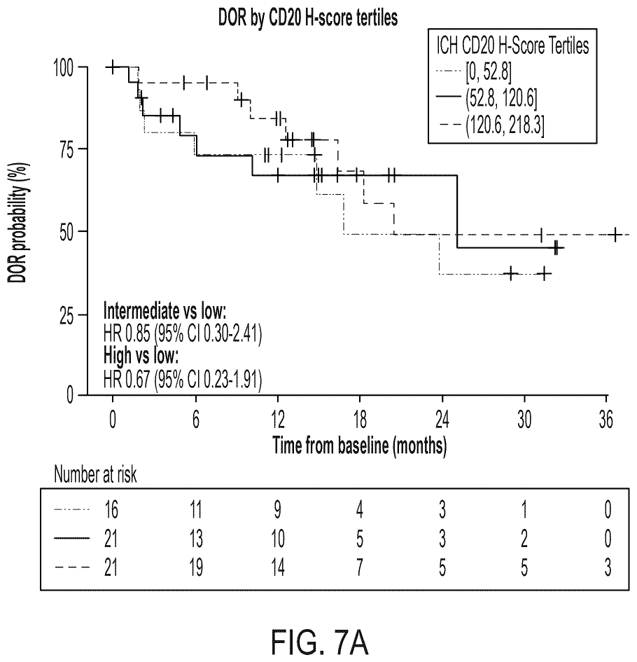

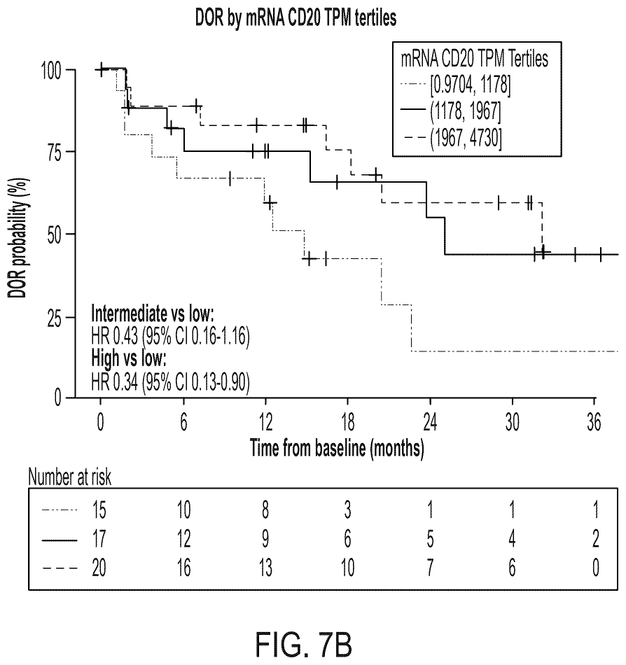

Tertile analysis in patients with FL showed that higher CD20 expression trended with longer duration of response (DOR), which was more pronounced with mRNA that IHC analysis (FIGS. 7A and 7B). Univariant analysis in patients with FL revealed a marginal association between baseline LDH level or time since last treatment, and CD20 expression, by H-score and mRNA TPM. Multivariate analysis in FL patients showed that mRNA CD20 TPM remained significantly associated with PFS when adjusted for refractory status to last treatment, biopsy age, LDH level, time since last treatment, and POD24 (progression of disease within 2 years) status at baseline, and that CD20 H-score was marginally associated with PFS, but its association decreased when adjusted for LDH level or time since last treatment.

Conclusions: This is one of the first studies to report the association of baseline CD20 expression with clinical response to odronextamab in R/R FL and DLBCL. The data suggest that patients can benefit from odronextamab regardless of baseline CD20 expression, irrespective of the analysis method used. Associations were observed between CD20 expression and DOR/PFS in FL.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Sequences
SEQ ID NO: 1
EVQLVESGGGLVQPGRSLRLSCVASGFTENDYAMHWVRQAPGKGLEWVSVISWNSDSIGYADSVKGRFTISRD
NAKNSLYLQMHSLRAEDTALYYCAKDNHYGSGSYYYYQYGMDVWGQGTTVTVSSASTKGPSVFPLAPCSRSTS
ESTAALGCLVKDYFPEPVTVSWNSGALTSGVHTFPAVLQSSGLYSLSSVVTVPSSSLGTKTYTCNVDHKPSNT
KVDKRVESKYGPPCPPCPAPPVAGPSVFLFPPKPKDTLMISRTPEVTCVVVDVSQEDPEVQFNWYVDGVEVHN
AKTKPREEQFNSTYRVVSVLTVLHQDWLNGKEYKCKVSNKGLPSSIEKTISKAKGQPREPQVYTLPPSQEEMT
KNQVSLTCLVKGFYPSDIAVEWESNGQPENNYKTTPPVLDSDGSFFLYSRLTVDKSRWQEGNVFSCSVMHEAL
HNHYTQKSLSLSLGK
SEQ ID NO: 2
EVQLVESGGGLVQPGRSLRLSCAASGFTEDDYTMHWVRQAPGKGLEWVSGISWNSGSIGYADSVKGRFTISRD
NAKKSLYLQMNSLRAEDTALYYCAKDNSGYGHYYYGMDVWGQGTTVTVASASTKGPSVFPLAPCSRSTSESTA
ALGCLVKDYFPEPVTVSWNSGALTSGVHTFPAVLQSSGLYSLSSVVTVPSSSLGTKTYTCNVDHKPSNTKVDK
RVESKYGPPCPPCPAPPVAGPSVELFPPKPKDTLMISRTPEVTCVVVDVSQEDPEVQFNWYVDGVEVHNAKTK
PREEQFNSTYRVVSVLTVLHQDWLNGKEYKCKVSNKGLPSSIEKTISKAKGQPREPQVYTLPPSQEEMTKNQV
SLTCLVKGFYPSDIAVEWESNGQPENNYKTTPPVLDSDGSFFLYSRLTVDKSRWQEGNVFSCSVMHEALHNRE
TQKSLSLSLGK
SEQ ID NO: 3
EIVMTQSPATLSVSPGERATLSCRASQSVSSNLAWYQQKPGQAPRLLIYGASTRATGIPARFSGSGSGTEFTL
TISSLQSEDFAVYYCQHYINWPLTFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEVTHQGLSSPVTKSENRGEC
SEQ ID NO: 4
EVQLVESGGGLVQPGRSLRLSCVASGFTENDYAMHWVRQAPGKGLEWVSVISWNSDSIGYADSVKGRFTISRD
NAKNSLYLQMHSLRAEDTALYYCAKDNHYGSGSYYYYQYGMDVWGQGTTVTVSS
SEQ ID NO: 5
EVQLVESGGGLVQPGRSLRLSCAASGFTFDDYTMHWVRQAPGKGLEWVSGISWNSGSIGYADSVKGRFTISRD
NAKKSLYLQMNSLRAEDTALYYCAKDNSGYGHYYYGMDVWGQGTTVTVAS
SEQ ID NO: 6
EIVMTQSPATLSVSPGERATLSCRASQSVSSNLAWYQQKPGQAPRLLIYGASTRATGIPARFSGSGSGTEFTL
TISSLQSEDFAVYYCQHYINWPLTFGGGTKVEIKR
SEQ ID NO: 7
GFTENDYA
SEQ ID NO: 8
ISWNSDSI
SEQ ID NO: 9
AKDNHYGSGSYYYYQYGMDV
SEQ ID NO: 10
GFTEDDYT
SEQ ID NO: 11
ISWNSGSI
SEQ ID NO: 12
AKDNSGYGHYYYGMDV
SEQ ID NO: 13
QSVSSN
SEQ ID NO: 14
GAS
SEQ ID NO: 15
QHYINWPLT
SEQ ID NO: 16
ASTKGPSVFPLAPCSRSTSESTAALGCLVKDYFPEPVTVSWNSGALTSGVHTFPAVLQSSGLYSLSSVVTVPS
SSLGTKTYTCNVDHKPSNTKVDKRVESKYGPPCPPCPAPPVAGPSVFLFPPKPKDTLMISRTPEVTCVVVDVS
QEDPEVQFNWYVDGVEVHNAKTKPREEQFNSTYRVVSVLTVLHQDWLNGKEYKCKVSNKGLPSSIEKTISKAK
GQPREPQVYTLPPSQEEMTKNQVSLTCLVKGFYPSDIAVEWESNGQPENNYKTTPPVLDSDGSFFLYSRLTVD
KSRWQEGNVFSCSVMHEALHNHYTQKSLSLSLGK
SEQ ID NO: 17
ASTKGPSVFPLAPCSRSTSESTAALGCLVKDYFPEPVTVSWNSGALTSGVHTFPAVLQSSGLYSLSSVVTVPS
SSLGTKTYTCNVDHKPSNTKVDKRVESKYGPPCPPCPAPPVAGPSVELFPPKPKDTLMISRTPEVTCVVVDVS
QEDPEVQFNWYVDGVEVHNAKTKPREEQFNSTYRVVSVLTVLHQDWLNGKEYKCKVSNKGLPSSIEKTISKAK
GQPREPQVYTLPPSQEEMTKNQVSLTCLVKGFYPSDIAVEWESNGQPENNYKTTPPVLDSDGSFFLYSRLTVD
KSRWQEGNVFSCSVMHEALHNRFTQKSLSLSLGK
SEQ ID NO: 18
ASTKGPSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPPVAGPSVFLFPPKPKDTLMISRTPEVTCVVV
DVSQEDPEVQFNWYVDGVEVHNAKTKPREEQFNSTYRVVSVLTVLHQDWLNGKEYKCKVSNKGLPSSIEKTIS
KAKGQPREPQVYTLPPSRDELTKNQVSLTCLVKGFYPSDIAVEWESNGQPENNYKTTPPVLDSDGSFFLYSKL
TVDKSRWQQGNVFSCSVMHEALHNHYTQKSLSLSPGK
SEQ ID NO: 19
ASTKGPSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPPVAGPSVELFPPKPKDTLMISRTPEVTCVVV
DVSQEDPEVQFNWYVDGVEVHNAKTKPREEQFNSTYRVVSVLTVLHQDWINGKEYKCKVSNKGLPSSIEKTIS
KAKGQPREPQVYTLPPSRDELTKNQVSLTCLVKGFYPSDIAVEWESNGQPENNYKTTPPVLDSDGSFFLYSKL
TVDKSRWQQGNVFSCSVMHEALHNRFTQKSLSLSPGK
*****

1. A method of treating lymphoma comprising administering to a subject in need thereof a bispecific antibody comprising a first antigen-binding region that binds human CD20 and a second antigen-binding region that binds human CD3, wherein the subject is selected on the basis of exhibiting a decreased level of circulating tumor (ct) DNA relative to a reference level of ctDNA following an initial period of treatment with the bispecific antibody.

2. A method of selecting a subject for treatment of lymphoma with a bispecific antibody comprising a first antigen-binding region that binds human CD20 and a second antigen-binding region that binds human CD3, the method comprising (a) measuring a level of circulating tumor (ct) DNA in the subject following an initial period of treatment with the bispecific antibody, and (b) comparing the measured level of ctDNA in the subject against a reference level of ctDNA, wherein if the measured level of ctDNA in the subject is less than the reference level of ctDNA, then continuing to administer the bispecific antibody to the subject during a maintenance period.

3. The method of claim 1, wherein the reference level of ctDNA is a baseline level of ctDNA measured prior to the initial period of treatment with the bispecific antibody.

4. The method of claim 1, wherein the initial period of treatment comprises four cycles of treatment, wherein each cycle comprises weekly administration of a dose of the bispecific antibody for three weeks.

5. The method of claim 4, wherein the weekly administration of the dose during cycle 1 comprises step-up dosing, and/or split dosing wherein two dose fractions of the dose are administered on consecutive days.

6. The method of claim 1, wherein the decreased level of ctDNA corresponds to minimal residual disease (MRD) negativity.

7. The method of claim 1, wherein the lymphoma is follicular lymphoma or diffuse large B-cell lymphoma.

8. The method of claim 1, wherein treating lymphoma comprises further administering the bispecific antibody during a maintenance period.

9. The method of claim 2, wherein the bispecific antibody is administered once every other week during the maintenance period.

10. A method of treating lymphoma comprising administering to a subject in need thereof a bispecific antibody comprising a first antigen-binding region that binds human CD20 and a second antigen-binding region that binds human CD3, wherein the subject is selected on the basis of exhibiting an absence of tumor protein p53 mutations, as measured by circulating tumor (ct) DNA.

11. The method of claim 10, wherein the lymphoma is follicular lymphoma or diffuse large B-cell lymphoma.

12. A method of treating diffuse large B-cell lymphoma (DLBCL) comprising administering to a subject in need thereof a bispecific antibody comprising a first antigen-binding region that binds human CD20 and a second antigen-binding region that binds human CD3, wherein the subject is selected on the basis of exhibiting genetic modifications corresponding to an EZB subtype, as measured by circulating tumor (ct) DNA.

13. The method of claim 12, wherein the DLBCL is a germinal-center B-cell-like subtype.

14. The method of claim 12, wherein the DLBCL is a non-germinal-center B-cell-like subtype.

15. The method of claim 1, wherein the subject has relapsed or refractory disease.

16. The method of claim 1, wherein the first antigen-binding region of the bispecific antibody comprises three heavy chain complementarity determining regions, HCDR1, HCDR2 and HCDR3 comprising the amino acid sequences of SEQ ID NO: 7, 8 and 9, respectively, and three light chain complementarity determining regions LCDR1, LCDR2 and LCDR3 comprising the amino acid sequences of SEQ ID NO: 13, 14 and 15, respectively, and wherein the second antigen-binding region of the bispecific antibody comprises three heavy chain complementarity determining regions, HCDR1, HCDR2 and HCDR3 comprising the amino acid sequences of SEQ ID NO: 10, 11 and 12, respectively, and three light chain complementarity determining regions LCDR1, LCDR2 and LCDR3 comprising the amino acid sequences of SEQ ID NO: 13, 14 and 15, respectively.

17. The method of claim 16, wherein the first antigen-binding region of the bispecific antibody comprises a heavy chain variable region (HCVR) comprising the amino acid sequence of SEQ ID NO: 4 and a light chain variable region (LCVR) comprising the amino acid sequence of SEQ ID NO: 6, and the second antigen-binding region of the bispecific antibody comprises a HCVR comprising the amino acid sequence of SEQ ID NO: 5 and a LCVR comprising the amino acid sequence of SEQ ID NO: 6.

18. The method of claim 17, wherein the bispecific antibody comprises a human IgG heavy chain constant region, optionally of isotype IgG1 or IgG4.

19. The method of claim 17, wherein the bispecific antibody comprises a first heavy chain comprising amino acid residues 1-452 of SEQ ID NO: 1 paired with a common light chain comprising the amino acid sequence of SEQ ID NO: 3, and a second heavy chain comprising amino acid residues 1-448 of SEQ ID NO: 2 paired with a common light chain comprising the amino acid sequence of SEQ ID NO: 3.

20. The method of claim 17, wherein the bispecific antibody comprises a first heavy chain comprising the amino acid sequence of SEQ ID NO: 1 paired with a common light chain comprising the amino acid sequence of SEQ ID NO: 3, and a second heavy chain comprising the amino acid sequence of SEQ ID NO: 2 paired with a common light chain comprising the amino acid sequence of SEQ ID NO: 3.

21. The method of claim 1, wherein the bispecific antibody is odronextamab.

22. The method of claim 1, wherein the subject has a detectable level of CD20 in a tumor cell sample prior to treatment with the bispecific antibody, as measured by mRNA expression and/or immunohistochemistry.

23. The method of claim 1, wherein the subject has a non-detectable level of CD20 in a tumor cell sample prior to treatment with the bispecific antibody, as measured by mRNA expression and/or immunohistochemistry.

24. A method of treating lymphoma comprising administering a bispecific CD20×CD3 antibody to a patient in need thereof, wherein the patient is predicted to respond to the therapy, and wherein the patient exhibits a level of circulating tumor DNA (ct DNA) that is indicative of minimal residual disease negativity.

25. A method of treating lymphoma comprising administering a bispecific CD20×CD3 antibody to a patient in need thereof, wherein the patient is predicted to respond to the therapy, and wherein the patient does not exhibit TP53 mutations.
